(19) United States
(12) Patent Application Publication
(10) Pub. No.: US 2014/0271772 A1
(43) Pub. Date: Sep. 18, 2014

(54) BIODEGRADABLE SUBCUTANEOUS IMPLANTS AND METHODS OF MAKING

(71) Applicants: BARRY J. MARGULIES, LUTHERVILLE, MD (US); JILL K. BADIN, OLNEY, MD (US); SOGAND FARTASH-NAINI, PARKVILLE, MD (US); THAMAR ETIENNE, REISTERSTOWN, MD (US); SARAH FARGIS, OAKTON, VA (US); AMANDEEP SAMRA, BALTIMORE, MD (US)

(72) Inventors: BARRY J. MARGULIES, LUTHERVILLE, MD (US); JILL K. BADIN, OLNEY, MD (US); SOGAND FARTASH-NAINI, PARKVILLE, MD (US); THAMAR ETIENNE, REISTERSTOWN, MD (US); SARAH FARGIS, OAKTON, VA (US); AMANDEEP SAMRA, BALTIMORE, MD (US)

(21) Appl. No.: 14/199,010

(22) Filed: Mar. 6, 2014

Related U.S. Application Data
(60) Provisional application No. 61/790,411, filed on Mar. 15, 2013.

(51) Int. Cl. A61K 9/00; A61K 31/522

(52) U.S. Cl. CPC A61K 9/0024; A61K 31/522
USPC 424/423; 514/263.37

(57) ABSTRACT

The present invention relates to a biodegradable implantable drug delivery device comprising a biodegradable polymeric material in combination with a therapeutic compound effective for the treatment of a member of the herpesvirus family, wherein the biodegradable implantable drug delivery device is fabricated by a novel method that provides for increased homogeneity and dispersity of the therapeutic compound impregnated within the biodegradable polymeric material.

### CROSS-REFERENCE TO RELATED APPLICATION

The present application claims priority to U.S. Provisional Patent Application No. 61/790,411 filed on Mar. 15, 2013, the contents of which are hereby incorporated by reference herein.

### STATEMENT REGARDING FEDERALLY SPONSORED RESEARCH

This invention was made with government support under Grant Number 1R15AI084069-01A2 awarded by the National Institutes of Health. The government has certain rights in the invention.”

### BACKGROUND THE INVENTION

1. Field of the Invention

The present invention relates to a delivery device, method of formulating such device and methods of using such device for the treatment of Human herpes simplex virus (HSV), and more specifically, to a biodegradable implant system for the controlled release of an effective drug for the treatment of HSV, varicella zoster virus (VZV), or feline herpesvirus-1 (FHV-1).

2. Description of Related Art

Herpesviruses are ubiquitous pathogens that infect many different animal species worldwide whose clinical manifestations can range from asymptomatic disease to life-threatening illness in immunocompromised hosts and neonates. (1, 2) The human herpesviruses HSV-1, HSV-2, and VZV are of particular clinical importance because of their frequency and the debilitating sequalae associated with their uncontrolled recurrences. HSV-1 is usually associated with orofacial infections and encephalitis and HSV-2 usually causes genital lesions. (3) HSV spreads from person to person via direct contact with infected secretions and enters its host through mucous membranes. (1) Once primary infection of the epithelial tissues has subsided, the virus is able to enter the sensory neurons and establish a life-long latent infection which allows some individuals to never show signs of disease. (4-6)

Human herpes simplex virus type-1 (HSV-1) is an alphaherpes virus in the group Simplexviridae. This is a widely studied virus that is used as a model virus for understanding herpesvirus replication and infection pathways. HSV-1 infects mucosal epithelia and dermal epithelial cells, causing lesions on the epithelium of the face, generally on the lips or nose. (1). Such lesions are typically termed cold sores or fever blisters. There are, however, other more serious diseases associated with HSV-1, including conjunctivitis, keratitis, hepatitis and encephalitis.

While estimates range, it is estimated that 80% of the adult population carries HSV-1, typically asymptomatically. (1) Primary infection usually occurs during childhood, and subsequent to the initial outbreak, the virus enters the peripheral nervous system, residing there permanently in a latent state of infection. The typical residence of latent HSV-1 is the trigeminal ganglia, which is the fifth cranial nerve arising from the pons, branching to enervate the face. (4) During the latent state, unincorporated viral DNA is present in the nucleus of the majority of the sensory nerve cells of the trigeminal ganglia, but no signs or symptoms of infection are present. (4)

Once primary infection of the epithelial tissues has subsided, the virus is able to enter the sensory neurons and establish a life-long latent infection which allows some individuals to never show signs of disease. However, many individuals do experience recurrent outbreaks which can be initiated through a variety of local and systematic stimuli such as physical or emotional stress, fever, UV light exposure, tissue damage or immune suppression. (3,7) Of these people who do see a recurrence, 5% have recurrence rates of at least one episode per month, 34% have at least one episode every two to eleven months, and 61% have at least one episode per year (3). Reactivation of the virus may be attributed to many factors, including burns, physiological and emotional stress, fever, hormonal changes, and exposure to ultraviolet light (3).

Several different treatments are available for combating infections with human herpes viruses. Therapies focus on either treatment of acute symptoms or suppression of the virus from reactivation. The main way to treat HSV-1 today is oral acyclovir (ACV), even though it is slowly and incompletely absorbed and has a low oral bioavailability of 10-30% (8) and a short in vivo half-life of 3 hours. This creates the need to administer multiple doses a day, which introduces the problem of patient compliance.

Despite the efficacy of ACV in the treatment of HSV-1, patient compliance and bioavailability are still major issues and, therefore, maintenance of the requisite levels of drug in the patient is a potential problem. U.S. Patent Publication No. 2008/0003250, describe a delivery system, developed by one of the present inventors, which includes a non-biodegradable silicone polymer implant containing ACV which provides for long-term drug delivery that has shown to be effective in suppressing HSV-1 reactivations in vitro and in vivo. Although this antiviral drug delivery system is effective at suppressing recurring HSV-1 outbreaks, it does require repeated surgical intervention for implant replacement.

Thus, it would be advantageous to provide an effective alternative for delivering an antiviral drug such as ACV that employs the use of a controlled release delivery device but without the disadvantages of the prior methods and systems for delivery. The ideal vehicle would release ACV at a steady and suppressive dose over an extended period of time, thereby obviating the problems with bioavailability, patient compliance, and the requirement for new prescriptions of oral ACV month after month, year after year or the need for removal/replacement of a non-biodegradable system.

### SUMMARY OF THE INVENTION

The present invention relates to the controlled release of at least one therapeutic compound from an implantable biodegradable polymeric substrate, wherein the therapeutic compound is evenly and homogeneously dissolved, distributed, dispersed and/or impregnated into the biodegradable polymeric material of the substrate.

In one aspect, the present invention relates to an implantable drug delivery device comprising a biodegradable polymeric material in combination with a therapeutic compound effective for the treatment of an alpha herpes virus in a mammal including members of the herpesvirus family such as feline or human herpesvirus. Preferably, the therapeutic compound is in an amount that will effectively treat HSV-1 and/or HSV 2 and reduce reactivation. Importantly, the implantable drug delivery device can effectively be positioned near the site of latent infection. The therapeutic compound may include any effective nucleoside that reduces reactivation of latent infections and/or controls the infection including but not limited to acyclovir (ACV), guanosine, valacyclovir, famiciclovir, penciclovir and functional analogues, functional equivalents or a combination thereof.

In another aspect, the present invention relates to a method for treating or controlling an alpha herpesvirus, the method comprising implanting into a subject, having the need for such treatment, the biodegradable implantable drug delivery device of the present invention and positioning such device at the local point of infection, or at the site pf observed clinical symptoms, such as the surface epithelium, mucus membrane or cutaneous layer on which the lesions are likely to appear. Notably when the infection is due to HSV, the local delivery may be positioned near or at the trigeminal ganglia or other nerves that enervate the facial epithelium.

The drug delivery device is preferably fabricated of a biodegradable polymeric material formed into a substrate that is sufficiently flexible for comfortable inclusion in a subject, yet sufficiently rigid for ease of insertion. Notably, the substrate can be fabricated into any geometric shape that provides sufficient surface area for inclusion and delivery of a therapeutic amount of an active agent found to exhibit antiherpes effectiveness. For example, the geometric shapes for implantation may include rods, bundle of multiple rods, disks, doughnut shapes, helical, elliptical, triangular or oval shapes. Preferably, rods have a diameter from about 1 to 3 mm and can be from about 5 to 20 mm long, and more preferably the diameter is about 2 mm with a length of about 15 mm. Disks can include structure with a thickness of from 1 to 3 mm with a diameter from about 3 mm to 8 mm. Notably, the time span for the degradability of the biodegradable polymeric material preferably does not exceed the diffusion rate of the therapeutic compound from the polymeric material.

Yet another aspect of the present invention relates to a method of fabricating a biodegradable drug delivery implant, the method comprising:
a. adding a biodegradable polymer to a polar aprotic solvent;
b. heating and agitating the solution until the biodegradable polymer is dissolved;
c. adding a therapeutic drug in an amount to form a homogeneous mixture, wherein the therapeutic drug is in an amount to form an implant comprising from 5% wt to 30% wt of the therapeutic drug;
d. adding formic acid in an amount to completely dissolve the biodegradable polymer and therapeutic drug to form a non-cloudy solution devoid of colloids;
e. evaporating the polar aprotic solvent and formic acid to form a dry solid wherein the therapeutic drug is essentially homogeneously dissolved or dispersed in the biodegradable polymer; and
f. melting, extruding, or compression molding the dry solid and forming the delivery device.

The polar aprotic solvent may be selected from the group consisting of tetrahydrofuran (THF), ethyl acetate (EtOAc), acetone, dimethylformamide (DMF) and acetonitrile (MeCN) and preferably acetonitrile.

A further aspect of the present invention relates biodegradable implant comprising ACV as therapeutic drug, wherein the biodegradable implant is fabricated by the following steps:
a. adding polycaprolactone (PCL) to a solution of acetonitrile, wherein the PCL is in an amount of about 3-6 g and preferable about 5 g and the acetonitrile is in an amount of about 15 to about 25 mL and preferably about 20 mL, or an average of about 1 g to about 4 mL of solvent;
b. heating and agitating the solution until the PCL is dissolved;
c. adding acyclovir (ACV) or penciclovir (PCV) in an amount to form an implant comprising from 5% wt to 30% wt of ACV or PCV to 95% to 70% of PCL;
d. adding formic acid in an amount of about 4 to 10 mL and preferably about 3 mL to 5 ml and in an amount to dissolve the PCL and ACV or PCV to form a clear solution with essentially no visible colloids;
e. evaporating the acetonitrile and formic acid to form a dry solid wherein the ACV or PCV compound is homogeneously dissolved and/or dispersed in the PCL and there is no longer any odor from formic acid; and
f. melting the dry solid and forming the delivery device.

In a still further aspect, the present invention relates to an implant according to the present description has a formulation of 5% to 40% therapeutic agent and 95% to 60% of the biodegradable polymer, such as, 5% ACV/95% PCL; 10% ACV/90% PCL; 15% ACV/85% PCL; 20% ACV/80% PCL; 25% ACV/75% PCL; 30% ACV/70% PCL; 35% ACV/65% PCL; and 40% ACV/60% PCL. In the alternative, the compound can be formulated using PCV, such as, 5% PCV/95% PCL; 10% PCV/90% PCL; 15% PCV/85% PCL; 20% PCV/80% PCL; 25% PCV/75% PCL; 30% PCV/70% PCL; 35% PCV/65% PCL; and 40% PCV/60% PCL.

Notably, specific amounts of the preferred biodegradable polymer may be omitted, and other biodegradable types added, or other therapeutic agents may be substituted, to adjust the therapeutic agent release rates.

In yet another aspect, the present invention relates to a method for treating and controlling HSV, the method comprising:
implanting subcutaneously a biodegradable polycaprolactone substrate fabricated by the methods of the present invention, wherein the substrate comprises a therapeutic compound for the treatment of HSV, and wherein the degradability of the biodegradable substrate is directly proportional to the amount of therapeutic compound released per unit time.

Therapeutic amounts of the therapeutic compound may be in the range from about 0.02 to 200 wg/day and more preferably from about 1 to 100 wg/day, and most preferably, from about 0.5 to about 2 wg/day. Unexpectedly, the dosage level per day to suppress HSV-1 is significantly lower than currently administered in oral dosage regimes.

Other features and advantages of the invention will be apparent from the following detailed description, drawings and claims.

### BRIEF DESCRIPTION OF THE FIGURES

FIG. 1 A-F show the steps of the Volatile Acid-Solvent Evaporation (VASE) of the present invention.

FIG. 2 shows results of scanning electron microscopy of ACV-containing implants produced by Suspension of Insoluble Drug (SID) versus the VASE method of the present invention.

FIG. 3 shows the ¹H nuclear magnetic resonance of PCL and ACV before and after VASE.

FIG. 4 show the melting point of PCL-ACV VASE versus untreated PCL and ACV.

FIG. 5 shows the ACV release from differently fabricated implants, that being, from the VASE method of the present invention using a biodegradable implant, an implant made from non-biodegradable silicone and the SID method of fabrication.

FIG. 6 illustrates photographs showing Vero cells 96 hours after either mock infection or HSV-1 infection. (A) Infected cells treated with PCL. (B) Infected cells treated with silicone-ACV implant. (C) Infected cells treated with a VASE implant. (D) Infected cells without treatment. (E) Uninfected cells without treatment. (F) Uninfected cells treated with a VASE implant.

FIG. 7 shows the average release kinetics of 70:30 PCL:ACV and 70:30 PCL:PCV VASE implants for first 15 days of drug release in PBS.

FIG. 8 shows the average release kinetics of 70:30 PCL:ACV and 70:30 PCL:PCV VASE implants from weeks 3 through 9 of drug release PBS.

FIG. 9 shows the results of a five day cell culture study showing average PCV release from different polymer:drug (polycaprolactone:penciclovir (PLC:PCV)) VASE implant ratios.

FIG. 10 shows the results of HSV-2 titers from infected Vero cells treated with different polymer:drug (polycaprolactone:penciclovir (PLC:PCV)) VASE implant ratios.

FIG. 11 shows the cytopathic effect in HSV-2-infected Vero cells treated with different polymer:drug (polycaprolactone:penciclovir (PLC:PCV)) VASE implant ratios.

### DETAILED DESCRIPTION OF THE INVENTION

### Definitions

The terms “subject,” “individual,” and “patient,” as used herein, are used interchangeably and refer to any subject, generally a mammal (e.g., human, canine, feline, equine, bovine, rodent, etc.), in which drug delivery is desired.

The term “therapeutically effective amount,” as used herein, is meant an amount of a therapeutic agent, or a rate of delivery of a therapeutic agent, effective to facilitate a desired therapeutic effect.

The terms “treatment” and “treating,” as used herein, refer to obtaining a desired pharmacologic and/or physiologic effect. The effect may be prophylactic in terms of complete or partial prevention of a disease or symptom thereof and/or may be therapeutic in terms of a partial or complete cure for a disease and/or adverse affect attributable to the disease.

The term “drug delivery device,” as used herein, is meant to encompass a device that can retain a quantity of drug and that facilitates movement of drug from the drug delivery device to a site external to the drug delivery device. “Drug delivery device” thus encompasses controlled drug release devices, as well as devices that release drug in an unpatterned (e.g., substantially unregulated) manner.

The term “controlled release,” as used herein, is meant to encompass release of substance (e.g., a drug) at a selected or otherwise controllable rate, interval, and/or amount. “Controlled release” thus encompasses, but is not necessarily limited to, substantially continuous delivery, patterned delivery (e.g., intermittent delivery over a period of time that is interrupted by regular or irregular time intervals), and delivery of a bolus of a selected substance (e.g., as a pre-determined, discrete amount of a substance over a relatively short period of time (e.g., a few seconds or minutes).

The term “biodegradable polymer” as used herein, means a polymer or polymers which degrade in vivo, and wherein erosion of the polymer or polymers over time occurs concurrent with or subsequent to release of the therapeutic agent. The terms “biodegradable” and “bioerodible” are used interchangeably herein. A biodegradable polymer may be a homopolymer, a copolymer, or a polymer comprising more than two different polymeric units.

The terms “drug formulation,” “formulation,” and “drug,” as used herein, are used interchangeably and meant to encompass any substance suitable for delivery to a subject, which substances can include pharmaceutically active drugs, as well as biocompatible substances that do not exhibit a pharmaceutical activity in and of themselves, but that provide for a desired effect at a treatment site, e.g., to flush or irrigate a treatment site (e.g., saline).

Notably, however, because the therapeutic compound is present at only low levels within the polymeric material, drug release is limited, at a predominantly constant rate, to low dose levels.

In one embodiment, polymers appropriate for use as matrix in the present delivery devices are biocompatible and biodegradable and slowing degrades during the period of drug release. Suitable for use as polymeric matrix in the present delivery devices are any biodegradable polymeric material and preferably polycaprolactone (PCL). Notably using PCL, there are no unwanted side effects, such as drug interactions or toxicity to the host, associated with ACV. Furthermore, PCL is considered safe and generally biocompatible and, most importantly, already approved by the US FDA for use in medical devices, including controlled release delivery.

The total weight of an implant is dependent on the amount of biodegradable polymer the activity or solubility of the therapeutic agent. Often, the dose of therapeutic agent is generally about 0.1 mg to about 200 mg of implant per dose. For example, an implant may weigh about 1 mg, about 3 mg, about 5 mg, about 8 mg, about 10 mg, about 100 mg, about 150 mg, about 175 mg, or about 200 mg, including the incorporated therapeutic agent.

A load of therapeutic agent associated with an implant will have a sustained release property or profile associated with it. For example, over the first 30 days after implantation, the implants described herein can release about 50 μg/day to about 200 μg/day. Over the lifetime of an implant, about 3 μg/day to about 40 μg/day can be released.

The proportions of the therapeutic agent, polymer and any other modifiers may be empirically determined by formulating several implant batches with varying average proportions. Release rates can be estimated, for example, using the infinite sink method, a weighed sample of the implants is added to a measured volume of a solution containing 0.9% NaCl in water, where the solution volume will be such that the therapeutic agent concentration after release is less than 5% of saturation. The mixture is maintained at 37° C. and stirred slowly. The appearance of the dissolved therapeutic agent as a function of time may be followed by various methods known in the art, such as spectrophotometrically, HPLC, mass spectroscopy, and the like until the absorbance becomes constant or until greater than 90% of the therapeutic agent has been released.

To prepare the delivery device, therapeutic compounds, preferably in particle form, are dispersed within the selected polymeric material using a protocol that is dictated primarily by the choice of polymer. Once the therapeutic particles are prepared and size-selected, they are dispersed uniformly within the selected polymer to achieve a volumetric loading which is appropriate for a porous network to form to provide for movement of the particles through the substrate and appropriate for the dosage level desired.

In a preferred embodiment of the invention, the particles are dispersed uniformly within the biodegradable polymeric material.

Shaping can be achieved by any conventional means, such as by extrusion, injection molding or by melt press. For example, the shape of the device may include cylindrical, bullet, elliptical, circular, bulbuous, loop or any other shape suited for placement in the biological environment.

In some embodiments, the particles or therapeutic compounds dissolved or dispersed in the biodegradable polymeric material may include one or more additional components that function to limit the rate of diffusion of the therapeutic compound from the substrate to the surrounding biological matrix, noting that such additional components are also biodegradable. In the alternative, the formulation may include one or more additional components that function to increase the rate of diffusion of the therapeutic compound from the substrate to the surrounding biological matrix and is biodegradable.

The present invention provides methods of treating a subject with an alphaherpes virus by administering a therapeutic compound via the biodegradable drug delivery device according to the invention. In operation, a substrate of the invention is implanted into a subject at or near the local point of infection, or at the site of observed clinical symptoms, such as the surface epithelium, mucus membrane or cutaneous layer on which the lesions are likely to appear. Preferably, if the virus is HSV-1, the delivery device is placed at or near the trigeminal ganglia, sacral, dorsal root ganglia or other nerves that enervate the facial epithelium or at the site of clinical signs or symptoms.

The biodegradable delivery device of the invention can be used alone or as an adjunct to other therapeutic regimen (e.g. oral or intravenous therapy, etc.).

Release characteristics of at least one therapeutic compound from the substrate into a surrounding medium may depend upon the level of loading and the degree of biodegradability. The present invention relates to a drug delivery device that provides for a controlled release of a therapeutic compound. In a preferred embodiment, the drug delivery device is fabricated from polycaprolactone in combination with at least one therapeutic drug compound, wherein the therapeutic compound is effective against HSV and other alpha-herpes viruses and is molecularly homogeneously suspended, dissolved, dispersed or impregnated within the polymer and the device provides for the controlled release of such a therapeutic compound into a surrounding physiological solvent. Preferably, the therapeutic compound is selected from acyclovir, valacyclovir, penciclovir and famciclovir.

ACV is a very effective, safe, and inexpensive drug capable of controlling alphaherpes infections and the present invention provides for a long-lived subcutaneous device that releases controlled levels of ACV. The ACV implant developed here is aimed at suppressing reactivation of an alpha virus including herpesviruses infecting both humans, such as HSV-1, and animals, such as feline herpes virus. However, it should be noted that other herpesviruses, including herpes simplex virus type-2 (HSV-2), the etiologic agent of genital herpes, and Varicella-Zoster virus (VZV), which causes chickenpox and shingles, are both susceptible to ACV therapy. (3)

The development of an ACV or PCV-polymeric implant capable of suppressing alpha herpesvirus reactivation would improve the quality of life for many patients. Additionally, this implant may also help prevent the spread of alpha herpesvirus infections, and allow for control of these infections in patients who are unwilling or incapable of compliance. A controlled release device for antiviral intervention that is popular and beneficial to an extremely large group of people may also lead to a change in the way other pharmaceuticals are delivered to patients.

### Examples

Exemplary, non-limiting examples and embodiments of the invention will now be described with reference to the figures.

Briefly, the present invention provides for rod-shaped implants constructed of polycaprolactone (PCL) mixed with ACV. Polycaprolactone (PCL) is a semi-crystalline, linear, resorbable aliphatic polyester that is subject to biodegradation and has been approved by the US Food and Drug Administration for use in a number of medical and drug delivery devices. (9, 10) PCL is considered suitable for long term drug delivery due to its high permeability to many drugs, non-toxic properties, and slow degradation rate (approximately 2-4 years). (11) Also, this polymer has been used extensively to construct biodegradable microspheres for the long-term delivery of drugs and proteins, such as taxol, heparin, naproxen, and insulin. (11, 12) The implants were designed from a homogenous mixture of PCL and ACV that was obtained via a novel solvent evaporation method. This residual drug:polymer mixture hardened into large pieces, and the resultant chunks were transferred to a 25 mL Combitip and heated in a hot water bath until they melted into a viscous mixture. The viscous mixture was then extruded through the Combitip into 12-gauge steel needles and allowed to cast into long rods with a diameter of 2.1 mm. Once the rod-shaped implants were removed from the needles, they were cut to 15 mm lengths.

Materials and Methods

Implant Development

Reviewing FIG. 1 the novel solvent VASE methodology of the present invention is illustrated wherein, (A) PCL is added to acetonitrile and (B) PCL dissolves with heat and constant agitation. (C) Once the PCL is dissolved, ACV is added to the solution. The solution turns white because the ACV forms a colloidal suspension. (D) Formic acid is added to the solution. The solution turns back to clear because the ACV dissolves in solution so completely that no colloids are now included. (E) The acetonitrile and formic acid are allowed to evaporate overnight. The result is a dry solid of molecularly homogeneous PCL and ACV. (F) This mixture is melted and plunged into a hollow needle. After it solidifies overnight, the mixture is extruded and cut into a 15 mm (length) by 2 mm (diameter) rod.

Determination of Whether the Formic Acid Treatment Compromised the Integrity of the PCL Polymer.

Gel permeation chromatography of PCL before and after implant development. GPC measures the molecular weight of the polymer and Table 1 shows the results. The samples were solvated to approximately 10 mg/mL using complete gel permeation chromatography (GPC) solvent which was 94% tetrahydrofuran (THF), 5% dimethyl sulfoxide (DMSO), 1% piperidine and a trace amount of butylated hydroxytoluene (BHT). 100 μL as a bolus was injected for analysis into a GPC instrument (Waters, inc.) using an autosampler at 1 mL/min and separated using 3 THF Styragel columns in series (37.8×300 mm). The molecular weight was determined via interpolation using polystyrene standards. Both SID and VASE treatments slightly reduced the number average molecular weight (Mn) of PCL (less than 10%). Notably, the SID method does not include the formic acid addition so the drug is maintained in a colloidal suspension throughout. The weight average molecular weight (Mw) and polydispersity index (PDI) were also insignificantly changed (less than 8% reduction in Mw). Formic acid did not significantly alter the Mn and Mw of PCL; SID v. VASE treatment resulted in a 1% reduction in Mn and a 5% reduction in Mw, small differences probably caused by the formic acid. These minor molecular weight shifts prove that VASE treatment had little to no effect on the integrity of the average polymer chain length.

TABLE 1

| | PCL only | SID | VASE |
|---|---|---|---|
| Mn | 18294 | 17264 | 17105 |
| Mw | 25656 | 25147 | 23772 |
| PDI | 1.402412 | 1.45663 | 1.389758 |

Determining the Homogeneity of the Implants

Scanning electron microscopy was used to compare ACV-containing implants produced by two different methods, that being, (S)uspension of (I)nsoluble (D)rug methodology v. (V)olatile (A)cid (S)olvent (E)vaporation methodology of the present claimed invention. Cross-sections of both types of implants were compared by scanning electron microscopy to ascertain whether there is a visual difference between the nonhomogenous SID implants and the molecularly homogeneous VASE implants. Samples were attached to aluminum stubs via carbon sticky tabs and coated with 20 nm of AuPd. Stubs were viewed and digital images were captured on a Leo 1530 FESEM. FIG. 2 (A) is a cross-section of SID implant where the crystallized ACV is highly visible (circles) (B) is a cross-section of a VASE implant, where homogeneity is established due to the absence of these ACV crystals and the uniform appearance of die implant.

¹H nuclear magnetic resonance of PCL and ACV before and after VASE.

¹H NMR investigates the change in the chemical structure of the drug and the polymer. Samples of untreated polycaprolactone (PCL), acyclovir (ACV), and VASE implant were dissolved in 50:50 dimethyl sulfoxide-d6 (DMSO) and acetonitrile-d3 (CD₃CN) in NMR tubes; tetramthylsilane (TMS) was added as an internal standard. ¹H NMR was carried out on a Jeol NMR spectrometer ECS-400. Peaks associated with each molecule ((PCL), Table 2; ACV Table 2) were compared before VASE and after VASE. While it appears that each chemical shift (in ppm) matches pre- and post-treatment, these data were graphed to demonstrate the identities as shown in FIG. 3. The graph in FIG. 3 shows that the equation of the best-fit line creates a near-perfect diagonal with a slope of about 1 and an R-squared fit of 1. The slope demonstrates that for each peak in the untreated material, there is an identical peak in the VASE-treated material. Together, Table 2 and FIG. 3 show that VASE treatment (formic acid treatment, in particular) does not change the native PCL and ACV, presumably allowing them to keep their useful properties.

TABLE 2

| | Chemical Shift in ¹H NMR (ppm) | |
|---|---|---|
| | Pretreatment | VASE |
| TMS | 0 | 0 |
| PCL | 1.323 | 1.323 |
| | 1.3471 | 1.3471 |
| | 1.5418 | 1.5418 |
| | 1.5613 | 1.5613 |
| | 1.5796 | 1.5796 |
| | 1.5957 | 1.5957 |
| | 1.6129 | 1.6129 |
| | 2.2566 | 2.2555 |
| | 2.2749 | 2.2749 |
| | 2.2933 | 2.2933 |
| | 3.4124 | 3.4124 |
| | 3.6163 | 3.6163 |
| | 3.6278 | 3.6289 |
| | 3.984 | 3.984 |
| | 4.0001 | 4.0001 |
| | 4.0172 | 4.0172 |
| | 4.1192 | 4.1192 |
| ACV | 3.5006 | 3.4983 |
| | 3.5075 | 3.5052 |
| | 4.4846 | 4.4852 |
| | 5.3609 | 5.3586 |
| | 6.3529 | 6.3609 |
| | 7.7252 | 7.7263 |
| | 10.5924 | 10.5351 |

FIG. 4 rising Differential Scanning Calorimetry shows no difference between pre- and post-treatment, to Tm, of polymer and drug. Display of data match the same methodology shown in FIG. 3.

In Vitro Release Kinetics

FIG. 5 shows the ACV release from implants over 8 weeks. VASE implants were placed into 6-well tissue culture plate with 7 mL of PBS at 37° C., 5% CO₂ in a humidified, environment. Every 2 d hr for 14 days, each implant, was moved into fresh PBS (FIG. 5A), then the collection was made in the same way every week for the next 6 weeks (FIG. 5B). High-performance liquid chromatography quantifying (HPLC) was used to quantify ACV in each sample. Each sample (50 μL) was prepared for analysis by dilution with 450 μL acetonitrile and analyzed on a Waters system equipped with Luna HILIC 3 micron 15×100 nm column under isocratic conditions running 90% acetonitrile/10% formic acid (0.1%) as the mobile phase. Values were calculated against ACV standards run simultaneously. Release of ACV (μg) was obtained each day from the uncovered rods for the first 2 weeks and then weekly for the remaining 6 weeks. FIG. 5A shows the release kinetics of ACV from the 3 types of implants for the first 14 days. There is a visible constant near zero-order release with the silicone implant, but VASE and SID implants did not demonstrate such release in the first 14 days. FIG. 5B highlights the drug release from weeks 3-8. It is evident that implant formulated by the VASE method of the present invention provided for continuous release at fairly uniform amount during the testing period.

Determining In Vitro Release Kinetics with Vero Cells

To test the efficacy and toxicity of the VASE implants, Vero cells were plated and different kinds of implants were placed in each well. Infected cells were treated with PCL implants (FIG. 6A), silicone implants (FIG. 6B), and VASE implants (FIG. 6C). Also, infected cells without any implant treatment (FIG. 6D) were compared to uninfected cells treated without treatment (FIG. 6E) and with VASE implants (FIG. 6F) in order to determine the possible toxicity of the VASE implants. The silicone (MED-4705, Nusil) implants also contain ACV, and were developed in the lab before the use of PCL and effectively prevent infection. The cells were infected with 1.8×10¹ pfu of HSV-1 per well, and pictures of each of the wells were taken four days post-infection. The VASE implants were shown to have antiviral efficacy, indicated by normal cell growth, and were nontoxic to the cells.

Determining Drug Concentration in the Implants

Three implants of each drug type were created using Volatile Acid Solvent Evaporation (VASE) method followed by sterilization through subsequent 70% ethanol and PBS submersion. Implants were left to dry overnight to remove residual ethanol and PBS. Each replicate was placed in a separate well on a 6-well plate. PBS (7 mL) was added to each well. The plate was incubated at 37° C. with 5% CO₂ in a humidified chamber. Every 24 hours, 1 mL of PBS was removed and stored at −20° C. for HPLC analysis. The remaining 6 mL of PBS was discarded as waste and 7 mL fresh PBS was added to wells; this process continued for the first 15 days. For HPLC, 150 μl of sample was mixed with 1350 μl acetonitrile and analyzed under isocratic 90% acetonitrile/10% formic acid (0.1%) on a 3μ HILIC column at 0.4 mL/min. This method was used to determine the drug concentration in each sample. The average of the three replicates was calculated and displayed in FIG. 7. Initially, acyclovir-polycaprolactone (ACV-PCL) implants exhibit maximum drug release on day 1, while the peak amount of drug released from the penciclovir-polycaprolactone VASE (PCV-PCL VASE) implant was day 2. After the peak release days of both drug implant types, there was no significant difference in the amount of drug released. Similarly, the amount of drug delivered by both implant types decreased over time.

The 70:30 PCL:ACV and 70:30 PCL:PCV VASE implants were tested to determine the drug release from weeks 3 through 9. Implants from FIG. 7 were followed past the initial 15 days. Following day 15, 1 mL PBS was collected using the exact procedure as described in FIG. 7, except collection was performed weekly instead of daily. HPLC analysis was conducted exactly as described in FIG. 7. Initially (week 3) there was no significant difference between the amounts of drug delivered between the two types of implants. However, after week 6, as shown in FIG. 8, PCV was released at a faster rate while the release rate of the 70:30 PCL:ACV implant remained relatively steady.

FIG. 9 shows a five day cell culture study showing average penciclovir (PCV) release from different polymer:drug (PCV) implant ratios. Implants containing varying concentrations of PCL:PCV were manufactured in the ratios of 70:30, 75:25, 80:20, 85:15, 90:10, 95:5, 100:0 (w:w). A 24-well plate was prepared with 1.6×10⁵ Vero cells per well in 1 mL complete Dulbecco's modified Eagle's medium (DMEM) and grown overnight in a 37° C. incubator at 5% CO₂. All the medium was removed 24 hr. later, then replaced with fresh DMEM. A single implant was placed in its own separate well; control wells received no implant. The next day, the medium was again collected and replaced, and each well was either left uninfected or infected with 100 pfu of HSV-2 (MS). The same medium and collection process was repeated for the next three days. The results showed that the 75:25 implant (containing the second highest drug load) delivered the most drug, which certainly does not agree with the norm wherein we would expect that the implant with the highest drug load would deliver the highest level of drug.

HSV-2 titers from infected Vero cells treated with different polymer:drug (PCV) implant ratios. Medium from the samples in FIG. 9 was analyzed for the amount of virus produced. Vero cells (4×10⁶ per well) were seeded in 1 mL in each well of a 12-well tissue culture plate. After 24 hours, the medium was removed and 50 μl of each sample, such as shown in FIG. 9, was added to each well. During the absorption period, the plate was placed into incubator at 37° C. for one hour; the plate was subtly rocked every 10 min during the absorption period to ensure even distribution of virus. A methylcellulose overlay (1 mL) was added to each well and wells were incubated at 37° C. for three days. After three days, the medium was removed, crystal violet was added, and the visible number of plaques were counted. Samples from each implant's polymer:drug ratio were replicated three times. The averages of each replicate in a set were calculated and used in FIG. 10. As expected from the highest level of drug release, as shown in FIG. 9, the 75:25 implant showed the lowest viral titers, indicating the highest suppressive activity.

Images were taken 3 days post infection of cells from FIGS. 9 and 10 to show the degree of cytopathic effect (CPE). Cytopathic effect or cytopathogenic effect (abbreviated CPE) refers to damage to host cells during virus invasion. This damage is measurable by obtaining viral titers. Images were taken from multiple focal planes and compiled using Zerene Stacker software. Data from the plaque assays (FIG. 10) were corroborated, showing the lowest amount of CPE coming from the 75:25 PCL:PCV VASE implant and the most CPE from the cells treated with the 100% PCL implant.

It is recognized that the implants of the present invention has a potential for veterinary application. Feline herpesvirus-1 is a common viral pathogen of domestic cats worldwide that causes severe conjunctivitis, keratitis, corneal ulceration, and even mortality. (13) Penciclovir is an anti-herpetic drug that is commonly prescribed in eye drops to treat cats with feline herpesvirus-1 (FHV-1) since acyclovir has been shown to be toxic to cats; famciclovir, the orally dosed prodrug of penciclovir, can also be used. Thus, there is potential for these implants to cross over into the veterinary prescription market by serving as an alternative to oral dosing and as an effective suppressive therapy for cats infected with FHV-1.

### REFERENCES

The contents of all references cited herein are incorporated by reference herein for all purposes.
1. Brady R C, Bernstein D I. Treatment of herpes simplex virus infections. Antiviral Res. February 2004; 61(2):73-81.
2. Koelle D M, Corey L. Herpes simplex: insights on pathogenesis and possible vaccines. Annu Rev Med. 2008; 59:381-395.
3. Whitley R J, Roizman B. Herpes simplex virus infections. Lancet. May 12 2001; 357(9267):15131518.
4. Baringer J R, Swoveland P. Recovery of herpes-simplex virus from human trigeminal ganglions. N Engl J Med. Mar. 29 1973; 288(13):648-650.
5. Tien R D, Felsberg G J, Osumi A K. Herpesvirus infections of the CNS: MR findings. AJR Am J Roentgenol. July 1993; 161(1):167-176.
6. Knipe B R, ed Fields Virology: Lippincott Williams and Wilkins; 2001.
7. Strauss J H S E G. Viruses and Human Disease. Second ed: Elsevier Academic Press; 2008.
8. Whitley R I, Gnann J W, Jr. Acyclovir: a decade later. N Engl I Med. Sep. 10 1992; 327(11):782-789.
9. Woodruff M A, Hutmacher D W. The return of a forgotten polymer-Polycaprolactone in the 21st century. Prog Polym Sci. October 2010; 35(10): 1217-1256.
10. Kweon H, Yoo M K, Park I K, et al. A novel degradable polycaprolactone networks for tissue engineering. Biomaterials. February 2003; 24(5):801-808.
11. Sinha V R, Bansal K, Kaushik R, Kumria R, Trehan A. Poly-epsilon-caprolactone microspheres and nanospheres: an overview. Intl Pharm. Jun. 18 2004; 278(1): 1-23.
12. Jiao Y Y, Ubrich N, Hoffart V, et al. Preparation and characterization of heparin-loaded polymeric microparticles. Drug Dev Ind Pharm. September 2002; 28(8):1033-1041.
13. Hussein F T, Menashy R V, Field H J. Penciclovir is a potent inhibitor of feline herpesvirus-1 with susceptibility determined at the level of virus-encoded thymidine kinase. Antiviral Res. June 2008; 78(3):268-274.

That which is claimed is:

1. An implantable drug delivery device comprising:
a substrate having a geometric shape for implantation within a subject, wherein the substrate comprises a biodegradable polymeric material and at least one therapeutic compound, and wherein the therapeutic compound is homogeneously dissolved at a molecular level and dispersed throughout the entire substrate.

2. The implantable drug delivery device of claim 1, wherein the geometric shape is selected from the group consisting of a rod, bundle of multiple rods, disk, doughnut, helical, elliptical, triangular and oval.

3. The implantable drug delivery device of claim 2, wherein the therapeutic compound is acyclovir or penciclovir.

4. The implantable drug delivery device of claim 3, wherein the therapeutic compound is in an amount to deliver from about 50 μg/day to about 200 μg/day.

5. The implantable drug delivery device of claim 1, wherein the implantable drug delivery device is fabricated by the following steps:
adding the biodegradable polymeric material to a polar aprotic solvent;
heating and agitating the solution until the biodegradable polymeric material is dissolved;
adding the therapeutic compound in an amount to form a homogeneous mixture, wherein the therapeutic compound is in an amount to form an implant comprising from 5% wt to 40% wt of the therapeutic compound;
adding formic acid in an amount to completely dissolve the biodegradable polymeric material and therapeutic compound to form a non-cloudy solution devoid of colloids;
evaporating the polar aprotic solvent and formic acid to form a dry solid wherein the therapeutic compound is homogeneously dissolved or dispersed in the biodegradable polymeric material; and
melting, extruding, or compression molding the dry solid and forming the delivery device.

6. The implantable drug delivery device of claim 5, wherein the polar aprotic solvent is selected from the group consisting of tetrahydrofuran (THF), ethyl acetate (EtOAc), acetone, dimethylformamide (DMF) and acetonitrile (MeCN).

7. The implantable drug delivery device of claim 5, wherein the polar aprotic solvent is acetonitrile.

8. The implantable drug delivery device of claim 7, wherein the therapeutic compound is acyclovir or penciclovir.

9. The implantable drug delivery device of claim 8 wherein the biodegradable polymer is polycaprolactone.

10. The implantable drug delivery device of claim 9, wherein the implantable drug delivery device is fabricated by the following steps:
adding polycaprolactone (PCL) to a solution of acetonitrile, wherein the PCL is in an amount from about 1 to 5 g and the acetonitrile is in an amount from about 10 to 30 mL;
heating and agitating the solution until the PCL is dissolved;
adding acyclovir (ACV) or penciclovir (PCV) in an amount to form an implant comprising from 5% wt to 40% wt of ACV or PCV;
adding formic acid in an amount of from about 4 to about 10 mL and in an amount to completely dissolve the PCL and ACV to form a clear solution with essentially no visible colloid;
evaporating the acetonitrile and formic acid to form a dry solid wherein the ACV compound is perfectly homogeneously dissolved and/or dispersed in the PCL; and
melting the dry solid and forming the delivery device in a geometric shape for implantation in a subject.

11. The implantable drug delivery device of claim 10 comprising 5% ACV/95% PCL; 10% ACV/90% PCL; 15% ACV/85% PCL; 20% ACV/80% PCL; 25% ACV/75% PCL; 30% ACV/70% PCL; 35% ACV/65% PCL; or 40% ACV/60% PCL; 5% PCV/95% PCL; 10% PCV/90% PCL; 15% PCV/85% PCL; 20% PCV/80% PCL; 25% PCV/75% PCL; 30% PCV/70% PCL; 35% PCV/65% PCL; or 40% PCV/60% PCL;

12. The implantable drug delivery device of claim 10, wherein the implantable drug delivery device is to administered to a subject infected with an alphaherpes virus.

13. The implantable drug delivery device of claim 12, wherein the subject is infected with HSV-1, HSV 2, VZV or FHV-1.

14. The implantable drug delivery device of claim 7, wherein the therapeutic compound diffuses into surrounding biological environment in a controlled released manner.

15. A method for treating or controlling a herpesvirus, the method comprising:
implanting into a subject having the need for such treatment, an implantable drug delivery device formulated by the method according to claim 10.

16. The method of claim 15, further comprising implanting the drug delivery device at or near the site of latent infection or at the site of observed clinical symptoms.

17. The method of claim 10, wherein the geometric shape is selected from the group consisting of a rod, bundle of multiple rods, disk, doughnut, helical, elliptical, triangular and oval.

18. The method of claim 17, wherein the rod has a length of approximately 5 mm to 15 mm.
